(19) United States
(12) Patent Application Publication
(10) Pub. No.: US 2021/0030450 A1
(43) Pub. Date: Feb. 4, 2021

(54) METHOD AND IMPLANT FOR BONE LENGTHENING

(71) Applicant: ALEXANDER NASSONOV, Kyriat Shmona (IL)

(72) Inventor: ALEXANDER NASSONOV, Kyriat Shmona (IL)

(21) Appl. No.: 16/526,468

(22) Filed: Jul. 30, 2019

(51) Int. Cl. A61B 17/72; A61B 17/68

(52) U.S. Cl. CPC A61B 17/7216; A61B 2017/681

(57) ABSTRACT

A new bone lengthening method using a force generated by own energy of a patient to activate a system based on an intramedullary telescopic nail 1, inserted into a broken bone. An outer driving module 30 connected by a cord 29, spanning either knee or elbow or femur-hip joint, where one end of the cord 29 connected to an inner driving module of the telescopic nail inserted into a broken bone and another end of said cord connected to the outer driving module attached to the second bone. The said system converts a tensile force into an axial displacement of a distal bone segment of the broken bone.

It is important to attach the outer driver 30 to an appropriate spot of skeleton in consideration that the cord starts pulling anchor 31 by active or passive leg movements only after passing 80% of natural range of motion.

### FIELD OF THE INVENTION

The present invention relates generally to orthopedic methods and implants for healing of bone fractures, manipulating of said fractures and, consequently, a limb length. More particularly, the present invention is an internal lengthening implant based on an intramedullary telescopic nail (IMTN) capable of changing its length and a method to activate said lengthening.

### BACKGROUND OF THE INVENTION

In order to elongate the limb, surgeons currently use two sets of techniques:
External fixation devices such as Ilizarov and Taylor frames and similar systems. A procedure of placing the external frame fixation system is difficult. Said systems rise discomfort and present an unattractive appearance. Moreover, pins, anchored to the bone and protruding outside the skin to join the frame increase risk of infection and nerve injury.
Internal fixation devices, such as intramedullary telescopic nails (IMTN) with adjustable length. IMTNs are fully implantable and have no parts protruding out through patient's skin.

There are plenty of patents, describing such IMTNs and methods of bone lengthening, but only a few methods and nails like Bliskunov nail SU707580(1977), Fitbone nail of Baumgart and Betz (1989), Albizzia nail of Grammont and Guichet U.S. Pat. No. 5,074,882 (1991) and Precice nail U.S. Pat. No. 8,197,490 (2009) of Ellipse Technologies Inc. are used by surgeons.

The Bliskunov method of bone lengthening based on a link spanning a joint of two bones, where one of bones is broken, and one end of the link connected to the bone from one side of said joint and another to the IMTN inserted into the broken bone from the other side of the joint. The Bliskunov device for femur elongation comprises a drive, made of a rigid telescopic rod, introduced through grater and medium sciatic muscles and connected further to a wing of an iliac bone and to a ratchet inside the nail. Said link spans the femur-hip joint and activates the elongation process by leg movements.

Other configurations of the Bliskunov IMTN, intended to elongate humerus and tibia, were presented in SU1029958 and SU1731196. Both latter designs, instead of the rigid telescopic rod, use a cord, spanning elbow and ankle joints subsequently. Similar distraction mechanism for bone segment transport (not lengthening) using the cord, spanning knee joint, described in U.S. Pat. No. 6,059,755 of Horas and Erben. The Bliskunov and Horas, Erben methods have very complicated procedure of implantation and are susceptible to uncontrolled elongation.

To activate elongation step of the Albizzia IMTN patients performs painful and not comfortable twist movements of broken bones parts. Said twists are painful and harm a natural process of callus forming and consolidation. Said IMTNs are strong enough to allow walking with crutches.

Fitbone and Precice IMTNs avoid problems with high level of pain described above, because both use an outside source of energy and standard procedure of implantation, but not strong enough and then need to use wheelchair for several months.

All said telescopic nails, based on differently operated screw-nut pairs (SN-pairs), cannot bear loads generated by everyday routine activity of healthy people (real full weight bearing, not limited weight bearing provided by crutches or wheelchairs). During lengthening period (consisting of elongation and consolidation periods) the SN-pair serves two purposes: firstly, to generate the distraction force, to stretch muscle, tendons, and nerves; secondly, to support load generated by body weight and activity.

The patient using Fitbone and Precice IMTNs must be aware every moment not to put the load more than ˜20 kg per leg to avoid SN-pair's thread deformation and subsequently whole failure of the nail. The load per leg can be increased by using of special sorts of stainless steel for manufacturing, but at very high cost.

Modern internal telescopic nails interfere everyday life of patients for approximately one-year period because patients must use crutches or wheelchair. Patients using Precice and Fitbone nails, compare to Bliskunov and Albizzia, need additional time to return to normal life after treatment due to inability to walk within elongation period. Because of inability to walk without support patients must sacrifice about one year of their life to get taller.

### SUMMARY OF THE INVENTION

The object of the invention is to develop a method and an internal implant for bone lengthening by distraction osteogenesys decreasing a total time of lengthening process and improving the everyday life of patient by providing him ability of walking without crutches and wheelchair through elongation and consolidation periods of time. It will dramatically increase quality of patients' life.

The object of the present invention further is a new bone lengthening method and an implant using a force generated by own energy of a patient to activate an internal lengthening implant based on an intramedullary telescopic nail, inserted into to the broken bone. A cord spans knee, elbow or femur-hip joint. One end of the cord connected to an inner drive of the telescopic nail and another end to the outer drive attached to the second bone. The said system converts a tensile force, applied to the cord by movement of bones connected by said joint, into an axial displacement of a distal bone segment of the broken bone.

The object of the invention further is to develop a method and an internal implant based on Bliskunov method to activate lengthening by using patient's own force using a link spanning a joint. According to the invention, rather than the rigid rod, the cord spanned the femur-hip joint. One end of the cord connected to the outer driving module that attached to the hip bone. Another end of the cord being connected to the inner driving module of the implant, inserted into an intramedullary canal of femur bone. Many years and hundreds of surgeries shows that the patient can provide enough force to turn SN-pair that works well under load, i.e. trapezoidal or another suitable thread. Postoperative recovery is hastened with weight bearing being a crucial part of healing.

The outer driving module allows free movement of patient's limbs in everyday life activity. According to the first scenario, to stop said free movement and activate the lengthening process, the patient must reach the stroke limit of the cord. Then the cord pulls out parts of an inner driving module, converting linear shift into rotation of the nut, then further pushing out the distal portion of broken bone. It is important to attach the outer driving module to an appropriate spot of skeleton in consideration that the cord starts pulling anchor by active or passive leg movements just after passing 80% of natural range of motion (ROM) of the joint.

Another mode of operation of said outer driving module is including into said module a normally open latch, to allow free movement within the full range of motion. The latch can lock said limbs movements in any position by external magnetic force. To control a range of lengthening the novel implant has a tracking ratchet that repeatedly makes clicking noise after 0.25 mm of the lengthening.

The new method and implant have following advantages over existing methods and implants of bone lengthening:
It allows walking without crutches and improve everyday life of patients;
It decreases level of pain;
No need in special devices and help of trained personnel;

### BRIEF DESCRIPTIONS OF THE DRAWINGS

Advantages and features of the invention will be more understood in conjunction with the following explanations to the attached drawings.

FIG. 1 is an isometric view of the new IMTN body of the new implant.

FIG. 1A shows a partially cut view of said body.

FIG. 1B, FIG. 1C and FIG. 1D depict enlarged views of chosen parts of said body.

FIG. 2 is a view of a subassembly for less invasive connection of a male half of connector to the IMTN body.

FIG. 2A and FIG. 2B are a front and a section views of the male half of connector.

FIG. 2C is an enlarged view of a portion of said section view.

FIG. 3 depicts a view of inner parts of the IMTN body.

FIG. 3A shows a base-rod of the inner driving module with some parts of said driver.

FIG. 3B presents parts comprising a kinematic chain of the inner driving module converting the pulling force of the cord into rotation of the driving nut.

FIG. 3C depicts a driving axle.

FIG. 3D shows elements of a female part of connector made inside the base-rod.

FIG. 4 and FIG. 4A show a front and a section views of the outer driving module.

FIG. 4B shows an enlarged view of a portion of said section view.

FIG. 4C and FIG. 4D depicts views of a base and a pulley of the outer driving module.

FIG. 4E presents the outer driving module without a lid.

FIG. 5 is a cut view of another version of outer driving module intended to femur or tibia lengthening using the link spanning knee joint shown in a position that is ready to activate inner driving module.

FIG. 5A is a cut view of said second version of the outer driving module in a starting position.

FIG. 6 is a view of the femur-pelvis joint and the whole lengthening system.

FIG. 7A and FIG. 7B show a kinematic scheme of tibia and femur elongation using the knee joint and the outer driving module with magnetic lock.

FIG. 8A and FIG. 8B show a kinematic scheme of tibia and femur elongation using the knee joint and the second version of the outer driving module.

### DETAILED DESCRIPTION OF THE PREFERRED EMBODIMENTS

According to the invention, the new internal lengthening implant, comprising an intramedullary telescopic nail (FIG. 1, FIG. 1A) consisting of a bushing 1, having a proximal (upper) and a distal (opposite) portions, female thread 2 at the distal portion of the bushing and transverse holes 3A, 3B at the proximal portion; a rod 4 with transverse holes 5A,5B at the distal portion, placed inside of said bushing, whereas the bushing 1 and the rod 4 have means (D-shape connection, for instance) preventing its mutual rotation. A driving nut 6, which interacted with the thread 2, forms together with the bushing SN-pair to drive out said rod 4.

The inner driving module of this new IMTN (FIG. 1A, FIG. 3, FIG. 3B) consisting of a base-rod 7 (FIG. 1A, FIG. 3A) with a bore 8 and a groove 9, a driving axle 10 (FIG. 3C) having helical 11 and axial 12 cut-outs and lips 13 (FIG. 3C, FIG. 3B) entering said groove, a piston 14 and a compression spring (not shown) pushing said piston 14 inside the cut-out 12. Said driving axle 10 (FIG. 3) placed coaxially into the bore 8 and connected through helical cut 11 by a pin-drive 15 to two tugs 16 (FIG. 3B) placed inside slits 16A along the base rod 7.

The driving axle 10 has a shaft 17 interacted with a one-way clutch (not shown) affixed inside a proximal half 18 of coupling 19 (FIG. 1A, FIG. 1B) whereas the wrench 20 and flat spring 22 (FIG. 3) attached to a distal part 21 of said coupling 19. The clutch-nut 23 (FIG. 1B) with second one-way clutch inside (not shown), restricts counter rotation of the nut 6, attached to the proximal end of bushing thread 2. The clutch-nut 23 has crown like lumps 24 (FIG. 1B). When the part 21 rotates the flat spring 22 and lumps 24 generate together a clicking sound, indicating that the nut turned 90°.

The clutch-nut 23 and the driving axle 10 has grooves 25 and 26 (not shown) with elastic O-rings inside (not shown), sealing gaps between the bushing 1 and driving axle 10 and the wrench 20 and the clutch-nut 23 consequently, making sealed volume containing both said one directional clutches and the flat spring. It makes possible using more powerful clutches made of non-implantable grade steel. To protect or lubricate the thread 2 could be used additional O-ring (not shown) inside a groove 27 at the bushing's 1 distal end.

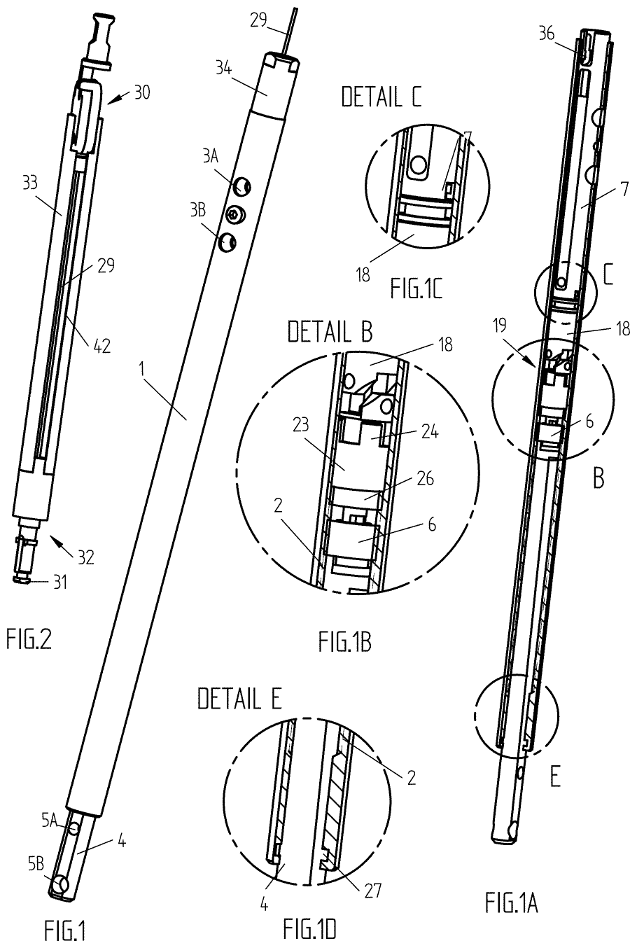

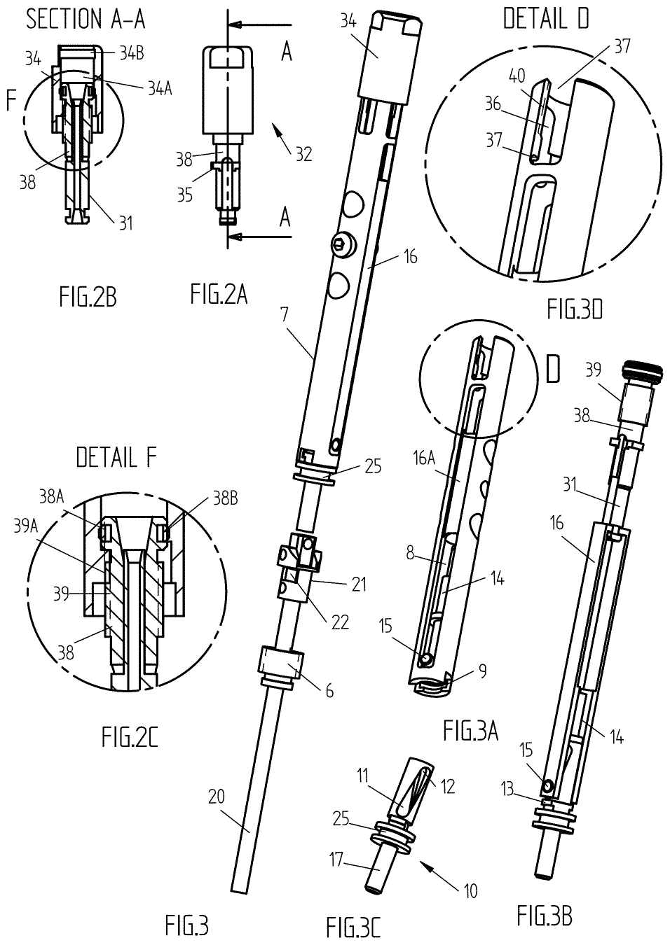

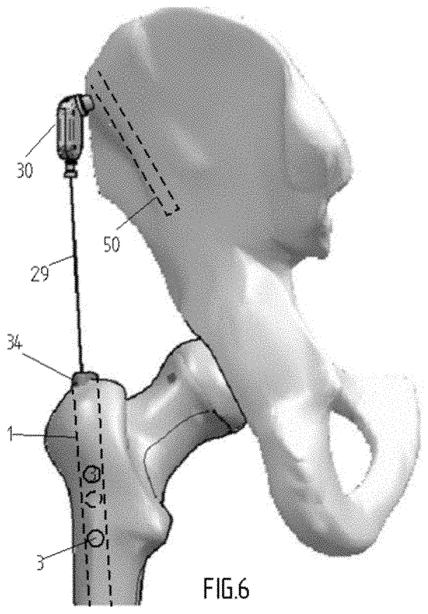

To incorporate the implant according to this invention, the IMTN body of the implant (FIG. 1A), must be placed inside the deliberately broken femur bone and affixed through holes 3A,3B and 5A,5B. To attach the connector 32 to the IMTN body, it is necessary to make passage through muscles from hip wing to piriform is of femur bone (FIG. 6). The outer driving module 30 (FIG. 1), connector 32 and tube 33, combined into temporarily assembly, has to be inserted through the passage and then attached, using the tube 33 as a wrench, by rotating of end cup 34 (FIG. 2B) to the proximal end of bushing 1.

The proximal end of the base-rod 7 (FIG. 3D) has a nest 36 with a thread 40 and a groove 37 forming together a cavity to accept of the connector 32 (FIG. 2A). The male part of the connector 32 (FIG. 2A) has an anchor 31 to catch and hold the tugs 16 (FIG. 3B) and the cord 29.

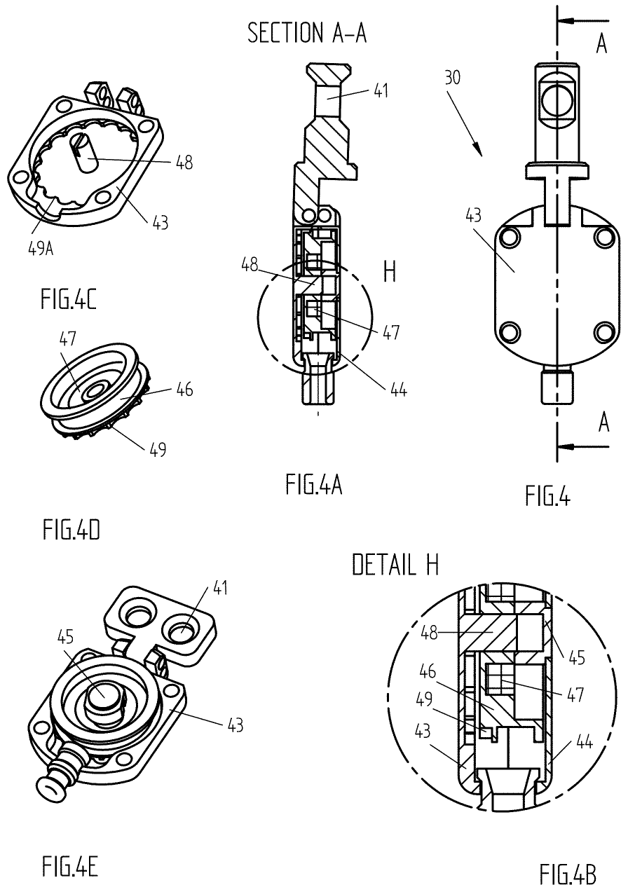

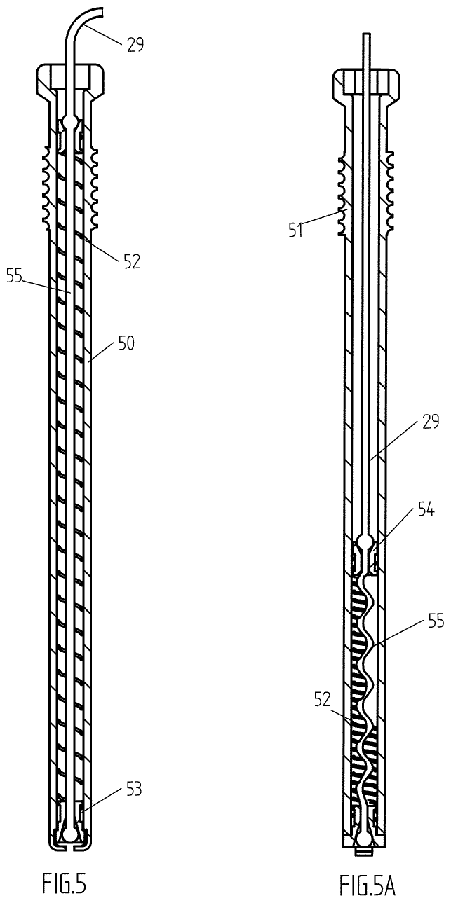

Then tube 33 is disassembled and moved out of the body of patient. The outer module 30 can be attached to a wing of pelvis through holes 41 (FIG. 4E, FIG. 6). To disconnect the tube 33 from the cord 29, a cut 42 is made along whole length of the tube 33. Finally, after the module 30 being connected to the wing, the system is ready to perform elongation of the femur bone.

The outer driving module 30 (FIG. 4) comprises a base 43, a lid 44, having ferromagnetic insert 45 and a pulley 46 with a magnet 47, affixed to said pulley 46. Said magnet 47 holds the pulley close to said insert 45.

The anchor 31 with means to catch and hold the tugs 16 and the cord 29 (t-like end of the anchor for the tugs and conical bore for the cord, for instance), whereas said anchor 31 being inserted in fork like base 38 with a groove 38A. After split ring 38B placed inside groove 38A it holds parts together in working position.

A threaded portion 39, interacting with thread 39A inside a cup-nut 34 having a bore 34A and a groove 34B inside said bore. The male half 32 (FIG. 2A) have two configurations: an initial, together with the tube-wrench 33, to insert and attach to the female half made inside the proximal end of the base rod 7 as it shown at FIG. 2A, FIG. 2B, and a working position, depicted at FIG. 3B, to support and provide ability to perform to and fro movement of the anchor 31 under force generated by the cord 29 and the compression spring inside the bore 8.

After inserting of the male half of connector 32 into the nest 36, the cup 34 by rotation retracts distally the fork 38 (FIG. 3B) until split ring 38B entered the groove 34B (FIG. 3B). The lip 35 guides fork 38 into the working position through the grooves 37. The split-ring 38B maintains the correct initial position of parts during inserting of the mail half 32 by tube-wrench 33 and insure that said parts remain in working position. In the working position the fork like base being placed proximally with the split-ring inside both 34B and 38A grooves. The threaded nest 40 supports implantation and removing of the IMTN by standard tools and technique.

The pulley 46 (FIG. 4B) connected by the cord 29 (not shown) to the anchor 31 and by a constant force spring (not shown) to an axle 48. The pulley, that can rotate and axially shift on the axle 48, holds the cord 29 under constant tension by the constant force spring (measuring tape mode). The constant force spring that placed inside of outer driving module is strong enough to retract said cord inside the module 30, but not stronger than compression spring inside the bore 8 of the base-rod 7 (FIG. 3A).

The spring-loaded pulley 46 of the outer module 30 allows free movement of the patient during everyday activity. According to the first preferred scenario, the patient puts designated end of barrel like permanent magnet on the skin opposing the outer module 30 in position that allows magnetic force pulls the pulley toward the base 43 (FIG. 4B). The pulley 46 and the base 43 have gear like protrusions 49 and 49A (FIG. 4C, FIG. 4D) that form normally open coupling with help of the magnet 47 and insert 45.

After the pulley shifts toward said protrusions 49A, it cannot rotate, and the patient can activate distraction by leg's movement. After 0.25 mm distraction was made (the spring 22 makes clicks to indicate a real rate of distraction), the patient removes said magnet and the pulley returns by force of magnet 47 to its normal position near the insert 45. To make disconnection of said protrusions easier, the patient can use opposite end of the magnet. Four times every day in the lengthening period the patient must lock position of the pulley to activate further the distraction mechanism by limb's movements.

Another mode of lengthening activation can start when the limb surpasses stroke of 80% of the normal range of movement of this joint. In this mode no need in the outer magnet to lock the pulley, just to perform movements exceeding said range. This latter mode of activation can be achieved by another embodiment of outer module (FIG. 5, FIG. 5A) as well. It could be assembled using large cannulated screw for instance by Smith&Nephew Inc. with enlarged inner bore or any other suitable large cannulated screw.

The screw 50 has the threaded portion 51 to attach to the bone, a coil extension spring 52 attached to the distal portion 53 of said screw and to the tug 54, wherein the cord 29 connected to the tug 54 as well and a second cord 55 connects said tug 54 and the distal portion 53, whereas all said elements placed inside said cannulated screw 50. The cord 55 has a slack to allow active or passive leg movements till 90% of natural range of motion (ROM) of the joint.

Kinematic schemes of tibia and femur elongation using the retraction module with the second version of outer driving module are presented at FIG. 8A and FIG. 8 B. The cord spans knee joint and connects IMTN and the outer module 50. The IMTN body could be inserted through the knee joint into medullary canal of femur or tibia and outer module 50 then could be inserted inside tibia or femur consequently, as shown at FIG. 8A, FIG. 8B. The module 50 intended to the femur lengthening using femur—hip joint (FIG. 6) could be placed inside pelvis wing as it shown at FIG. 6 by a dash line.

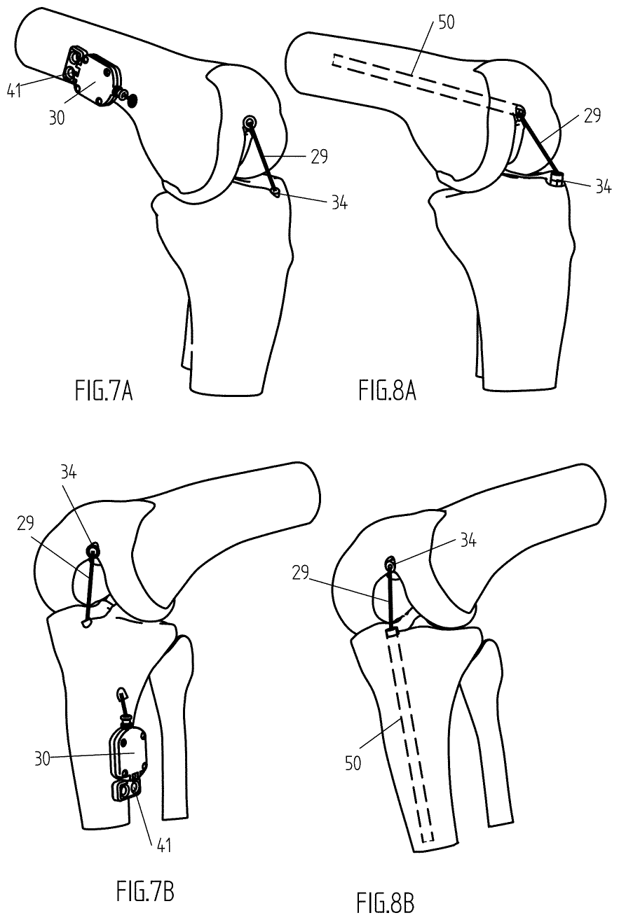

Movement of bones, forming a joint, pulls the cord 29 out of sleeve 50. The cord 29 connected to tug 54 pulls this part with the extension spring 52 out. After the second cord 55, following patient's leg movements, reaches his limit, determined by the size of the slack, the inner module starts lengthening. Since then the patient senses that limit is reached, there is a need to stop the movement and return to an initial position. The outer module 30 could be used with IMTN inserted through knee joint as well. It can be attached to the bone through the small cannulated screw (not shown) as it depicted at FIG. 7A, FIG. 7B.

It should be noted that the above description and the accompanying drawings are merely illustrative of the application of the principles of the present invention and are not limiting. Numerous other arrangements which embody the principles of the invention and which fall within its spirit and scope may be readily devised by those skilled in the art. The invention is not limited by the summary or scope of the invention as described in this application.

What is claimed is:

1. A method for bone lengthening by distraction osteogenesys using a force generated by own energy of a patient that moves bones, forming a joint, along anatomical pivot point, where at least one of said bones is broken, comprising following actions to generate said force:
placing a cord, spanning said joint, where one end of the cord connected to an inner driving module of intramedullary telescopic nail inserted into the broken bone and another end to an outer driving module attached to the second bone;
providing the inner driving module with ability to convert a tensile force, applied to the cord by movement of said bones, into an axial displacement of a distal bone segment of the broken bone;
selecting an appropriate spot of skeleton to attach the outer driving module in consideration that it allows free bones movement within at least 80% of natural range of movement of said joint and begins said conversion under active or passive leg movements after reaching over said limit;
providing said outer driving module with a normally open latch that allows free movement within the range of motion and can be locked by external magnetic force in any position within full range of motion.

2. A bone lengthening implant, comprising an intramedullary telescopic nail consisting of a bushing, having female thread at its distal portion and transverse holes at its proximal portion, a rod with transverse holes at distal portion, placed inside of said bushing within said threaded portion, whereas the bushing and the rod has means preventing mutual rotation, a driving nut, which interacted with said rod and thread; and the inner driving module affixed inside of the proximal portion of the bushing and connected to the outer driving module by the cord and a connector, that comprises a nest and grooves forming a female half of connector within the proximal end of inner driving module; a male part of the connector that consists of an anchor with means to grip the cord, whereas said anchor inserted in a fork like base, having a groove with a split ring inside, and a threaded portion, interacting with a thread inside a cup of said male part of connector having further a bore and a groove with snap ring inside of said groove.

3. The implant according to claim 2, wherein inner driving module consists of a bar with holes, drilled opposite the holes in the bushing, a bore, a coaxial groove at a distal end of said bore, a driving axle, having lips, entering said groove, and helical and axial cut-outs at its proximal portion; a piston and a spring, pushing said piston inside the driving axle; wherein said bore, driving axle, piston and spring, placed coaxially into the bore and connected by a pin, located inside said helical cut, to two tugs sited along the bar inside slits cut out along said bar and further connected to the anchor of the connector.

4. The implant according to claim 2, where the driving axle has a shaft interacting with a one-way clutch, affixed inside of a proximal half of cardan like coupling joint, where a distal half of said coupling joint has a wrench, placed inside the driving nut, and a second clutch, restricting counter rotation of said wrench, affixed inside of a clutch-nut that attached to the proximal end of bushing thread; wherein inside the distal half of coupling inserted a flat spring and the clutch-nut has crown like lumps, interacting with said flat spring; and the clutch-nut, the driving axle and the bushing have grooves, with elastic O-rings inside, placed between the wrench and the clutch-nut, the bushing and driving axle, the rod and the busing consequently.

5. The implant according to claim 2, where the outer driving module comprises a box consisting of a base with holes to attach said base to the bone and a lid, both made of non-magnetic material, the latter has ferromagnetic insert; a pulley with a magnet, affixed to said pulley, placed inside of said box on an axle that attached to the box, connected by the cord to the anchor of connector and by a constant force spring to the axle, whereas the base and the pulley have gear like protrusions to form normally open latch together; wherein the magnet has ring shape and placed coaxially around of said axle opposite the ferromagnetic insert and the cord is reeled up on the pulley and the constant force spring has enough length to allow free movement of bones within of natural range of the joint; whereas the latch consists further a magnet placed opposite the pulley out of patients body.

6. The implant according to claim 2, where the another version of outer driving module consists of a sleeve having a threaded portion at its proximal portion to attach to the bone, a coil extension spring inserted and attached to a distal portion of said sleeve, a slack-cord, placed inside of said spring; wherein said slack-cord, being connected to both spring's ends to constrain maximum stroke of said spring, connected further to the cord, linking the inner and outer driving module.
